(19) United States
(12) Patent Application Publication
(10) Pub. No.: US 2010/0263434 A1
(43) Pub. Date: Oct. 21, 2010

(54) GAS SENSOR

(75) Inventors: Shigeki Aoki, Yatomi-shi (JP); Daisuke Miyata, Nishikasugai-gun (JP); Makoto Hirasawa, Kasugai-shi (JP); Takayoshi Atsumi, Konan-shi (JP); Yasuhiro Fujita, Kaizu-shi (JP)

Correspondence Address: SUGHRUE-265550, 2100 PENNSYLVANIA AVE. NW, WASHINGTON, DC, 20037-3213, US

(73) Assignee: NGK SPARK PLUG CO., LTD., Nagoya-shi (JP)

(21) Appl. No.: 12/760,610

(22) Filed: Apr. 15, 2010

(30) Foreign Application Priority Data
Apr. 16, 2009 (JP) 2009-100049
May 22, 2009 (JP) 2009-124190

(51) Int. Cl. G01N 33/00; G01N 1/44

(52) U.S. Cl. 73/23.32

(57) ABSTRACT

A gas sensor extending along an axial direction, the gas sensor including: a housing having a through hole, the through hole including an expanding portion at a leading end thereof; a gas sensing element inserted into the housing, having a closed leading end, and provided with a sensor electrode on an outer surface of the leading end side thereof; a heater inserted into the gas sensor, and including a contact portion where the heater contacts an inner surface; an protector fixed on the leading end side of the housing and having an outer protector including an outer air hole and an inner protector being positioned within the outer protector and spaced apart from the outer protector in the radial direction; and an inner air vent provided between the inner protector and the leading end of the housing.

### BACKGROUND OF THE INVENTION

1. Field of the Invention

The present invention relates to a gas sensor for detecting a specific component (e.g., oxygen, or the like) contained in a gas to be measured (hereinafter also called a “target gas”); for instance, an exhaust gas from an internal combustion engine.

2. Description of the Related Art

A related gas sensor is known for detecting a specific component in a target gas, for instance detecting oxygen, or the like, in an exhaust gas emitted from an internal combustion engine. The gas sensing element used in such a related art gas sensor comprises a closed-end cylindrical solid electrolytic element having a closed leading end. The closed-end cylindrical solid electrolyte is provided with electrode layers on inter and outer surfaces thereof. In the related art gas sensor, for instance, an atmosphere is introduced as a reference gas into the inter surface of the gas sensing element, and a target gas is brought into contact with an outer surface of the gas sensing element. The gas sensor measures an electromotive force based on a gas concentration difference between the inner and outer of the gas sensing element, to thereby detect the concentration of the gas component.

The related art gas sensor has a metal housing holding the gas sensing element so that a detecting section formed at a leading end side of the gas sensing element protrudes from a leading end of the metal housing. The detecting section protruding from the leading end of the metal housing is covered with and protected by a protector having air holes. (see, for instance, JP-A-2005-326394).

With respect to the gas sensor, an amount of a material making up the gas sensing element, for instance, an amount of platinum used for the electrodes, is desirably reduced. Also, the entire length of the gas sensing element is desirably shortened in order to reduce manufacturing cost.

However, when the gas sensing element is shortened, the entirety or a portion of the gas detecting section provided at the leading end of the gas sensing element is accommodated in the housing instead of protruding to the outside from the leading end of the housing. As a result, responsiveness of the gas sensor is deteriorated due to shortening of the gas sensing element. Therefore, it has been difficult to sufficiently shorten the gas sensing element short without sacrificing responsiveness.

### SUMMARY

It is therefore an object of the present invention to provide a gas sensor having a shortened gas sensing element while maintaining good responsiveness.

The above object has been achieved by providing, in a first aspect of the present invention, a gas sensor extending along an axial direction from a rear end side to a leading end side thereof, the gas sensor comprising: a cylindrical housing having a through hole, the through hole including a radially expanding portion at a leading end thereof; a cylindrical gas sensing element inserted into the housing, having a closed leading end, and provided with a sensor electrode on an outer surface of the leading end side of the gas sensing element; a bar shaped heater inserted into the gas sensor, and including a contact portion where the heater contacts an inner surface opposite the outer surface of the leading end side of the gas sensing element, the contact portion and the leading end of the housing positioned in this order with respect to the axial direction from the rear end side to the leading end side; a protector fixed on the leading end side of the housing and having a cylindrical outer protector including an outer air hole and a cylindrical inner protector being positioned within the outer protector and spaced apart from the outer protector in the radial direction; and an inner air vent provided between the inner protector and the leading end of the housing, the inner air vent guiding gas entering a space between the outer and inner protectors into the through hole and into the inner protector.

According to the above first aspect, the heater contact portion of the gas sensing element is configured so as to be positioned on the rear end side relative to the leading end of the housing. The leading end side of the gas sensing element is a gas detecting portion which is heated by the heater so as to be sensitive to a gas component to be measured. Specifically, the entirety or a portion of a gas detecting portion is positioned at the rear end side (the interior) of the housing relative to the leading end of the housing. An expanding portion in which the through hole expands in diameter toward the leading end is formed in the housing, and an inner air vent for introducing a gas into the through hole and into the inner protector is provided between the leading end of the housing and the inner protector. The entire length of the gas sensing element can be shortened as compared to the related-art gas sensor. By employing this structure, a gas to be measured readily moves to a vicinity of the detecting portion of the gas sensing element inserted into the housing, so that deterioration of responsiveness can be prevented.

In a preferred embodiment, the detection electrode may be formed on an outer surface including the leading end of the gas sensing element (for example a solid electrolytic element) or on an outer surface of the leading end side without including the leading end of the gas sensing element. Further, in yet another preferred embodiment, the heater contact portion is in direct contact with an inner surface side opposing the detection electrode of the gas sensing element or remains in contact with the same by way of an intermediate member (e.g., a reference electrode, a protective layer, or the like). “The inner surface opposite the outer surface of the leading end side of the gas sensing element” refers to an inner surface crossing a normal drawn to an outer surface of the solid electrolytic element on which the detection electrode of the gas sensor is formed. The heater contact portion may also be a part or entirety of the leading edge of the rod-shaped heater, or a part of an outer periphery of the leading end side.

In yet another preferred embodiment, at least a portion of the inner air vent is positioned at a radially inner side of an imaginary line defined between a leading end of the expanding portion and a rear end of the expanding portion when the gas sensor is viewed in a cross section parallel to the axial direction. This structure allows a large amount of the gas to be measured which has passed through the space between the outer and inner protectors to quickly flow toward and into the expanding portion without substantial influence of the housing. Consequently, a large amount of the gas to be measured can quickly arrive at the vicinity of the heater contact portion of the gas sensing element (the gas detecting portion), so as to prevent deterioration of responsiveness.

In yet another preferred embodiment, at least a portion of the contact portion is positioned at a leading end side of an imaginary line defined between a leading end of the expanding portion and a rear end of the expanding portion when the gas sensor is viewed in a cross section parallel to the axial direction. This structure allows a large amount of the gas to be measured to quickly arrive at the vicinity of the heater contact portion of the gas sensing element (the gas detecting portion) by a gas flow running along an inner surface of the expanding portion, so as to prevent deterioration of responsiveness.

In yet another preferred embodiment, the rear end of the expanding portion and at least a portion of the contact portion are positioned in this order with respect to the axial direction from the rear end side to the leading end side when the gas sensor is viewed in a cross section parallel to the axial direction. This structure allows a large amount of the gas to be measured to more quickly arrive at the vicinity of the heater contact portion of the gas sensing element (the gas detecting portion) by the gas flow running along the inner surface of the expanding portion, so as to prevent deterioration of responsiveness.

In yet another preferred embodiment, the through hole within the expanding portion gradually expands in the radial direction along the axial direction from the rear end side to the leading end side. In this manner, a reduction in speed and amount of the gas to be measured flowing into the expanding portion before arriving in the vicinity of the heater contact portion (the gas detecting portion) of the gas sensing element, which would otherwise occur due to the expanding portion, can be prevented, to thus suppress further deterioration of responsiveness.

In a second aspect, the present invention provides a gas sensor extending along an axial direction from a rear end side to a leading end side thereof, the gas sensor comprising: a cylindrical housing having a through hole; a cylindrical gas sensor inserted into the housing, having a closed leading end, and provided with a sensor electrode on an outer surface of the leading end side of the gas sensing element; a bar shaped heater inserted into the gas sensor, and including a contact portion where the heater contacts an inner surface opposite the outer surface of the leading end side of the gas sensing element, the contact portion and the leading end of the housing positioned in this order with respect to the axial direction from the rear end side to the leading end side; a protector fixed on the leading end side of the housing and having a cylindrical outer protector including an outer air hole and a cylindrical inner protector being positioned within the outer protector and spaced apart from the outer protector in the radial direction; an inner air vent provided between the inner protector and the leading end of the housing, the inner air vent guiding gas entering a space between the outer and inner protector into the through hole and into the inner protector, wherein at least a portion of the inner air vent is provided at a radially inner side of a cross point between the through hole and the leading end of the housing when the gas sensor is viewed in a cross section parallel to the axial direction.

In a gas sensor having the foregoing configuration, the heater contact portion of the gas sensing element is configured so as to be positioned on the rear end side relative to the leading end of the housing. The leading end of the gas sensing element is a gas detecting portion which is heated by the heater so as to be sensitive to a gas component to be measured. Specifically, the entirety or portion of the gas detecting a portion is positioned at the rear end side (the interior) of the housing relative to the leading end of the housing. An inner air hole for introducing gas into the through vent and into the inner protector is provided between the leading end of the housing and the inner protector. At least a portion of the inner air vent is positioned at a radially inner side of the cross point between the through hole and the leading end of the housing. The entire length of the gas sensing element can be shortened as compared with the related art gas sensor. A large amount of gas which has passed through space between the outer and inner protectors quickly flows into the housing without substantial influence of the housing. Hence, the gas flow easily arrives at a vicinity of the detecting section of the gas sensing element inserted into the housing, so that deterioration of responsiveness can be prevented.

In a preferred embodiment of the first aspect or the second aspect, the gas sensor further comprises a gas flow hole provided at a leading end side of the inner protector. This configuration results in a smooth flow of gas circulating through the interior of the inner protector, so that deterioration of responsiveness can be prevented.

In yet another preferred embodiment of the first aspect or embodiment of the second aspect, the gas sensor further comprising the outer air hole provided at a leading end side of the leading end of the gas sensing element. This configuration prevents damage to the gas sensing element, which may otherwise be caused by the arrival of moisture, or the like, contained in the gas to be measured, such as an exhaust gas, at the gas sensing element.

According to the exemplary embodiments of the present invention, a gas sensor having a shortened gas sensing element can be obtained while also preventing deterioration of responsiveness.

### BRIEF DESCRIPTION OF THE DRAWINGS

FIG. 1 is a longitudinal cross sectional view showing a general overall configuration of a gas sensor of the first exemplary embodiment.

FIG. 2 is an expanded longitudinal cross sectional view showing a leading end portion of the gas sensor shown in FIG. 1.

FIG. 3 is a partial cross sectional-perspective view showing a protector of the gas sensor shown in FIG. 1.

FIG. 4 is an expanded longitudinal cross sectional view showing a leading end portion of the gas sensor of the second exemplary embodiment.

FIG. 5 is an expanded longitudinal cross sectional view showing a leading end portion of the gas sensor of the third exemplary embodiment.

FIG. 6 is a graph showing a result of investigation of a response characteristic of the embodiment and a response characteristic of a comparative example;

FIG. 7 is a graph showing a result of investigation of a response characteristic of the embodiment and a response characteristic of the comparative example;

FIG. 8 is a longitudinal cross sectional view showing, in an enlarged manner, a configuration of a principal section of a related art gas sensor;

FIG. 9 is a longitudinal cross sectional view showing, in an enlarged manner, the configuration of a principal section of the gas sensor of the comparative example;

FIG. 10 is a longitudinal cross sectional view showing, in an enlarged manner, a configuration of a principal section of a gas sensor of another comparative example;

FIG. 11 is a longitudinal cross sectional view showing, in an enlarged manner, a configuration of a principal section of a gas sensor of a second embodiment of the present invention; and

FIG. 12 is a lateral cross-sectional view taken along line A-A shown in FIG. 11.

### DETAILED DESCRIPTION OF THE PREFERRED EMBODIMENTS

The present invention will now be described in detail with reference to the drawings. However, the present invention should not be construed as being limited thereto.

### First Exemplary Embodiment

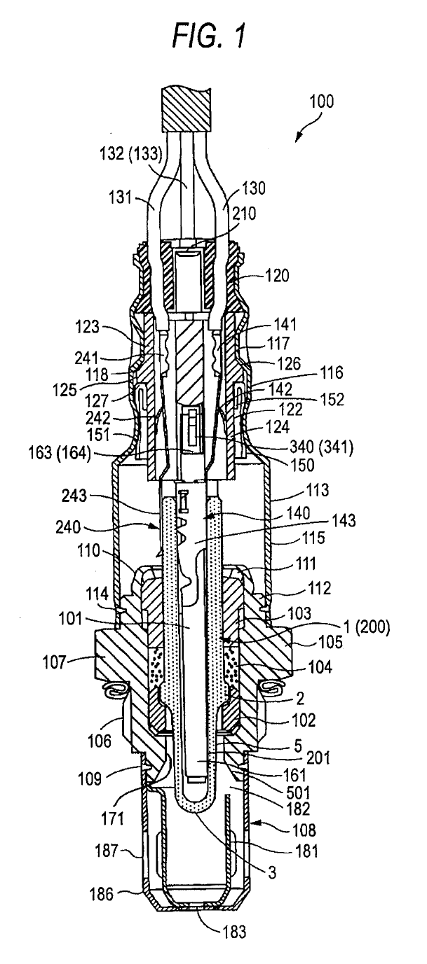

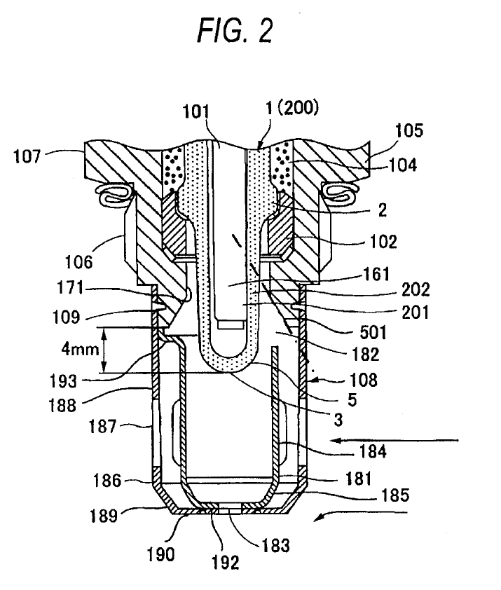

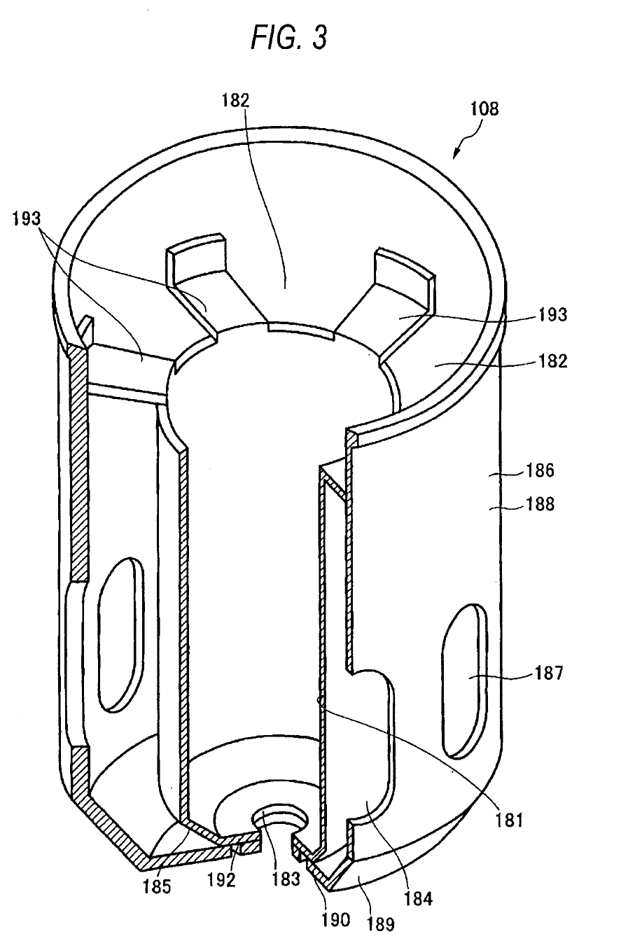

A first exemplary embodiment of the present invention is an oxygen sensor. FIG. 1 is a longitudinal cross sectional view showing a general overall configuration of an oxygen sensor of the first embodiment. FIG. 2 is an expanded longitudinal cross sectional view showing a leading end portion of the gas sensor shown in FIG. 1. FIG. 3 is a partial cross sectional-perspective view showing a protector of the gas sensor shown in FIG. 1.

As shown in FIG. 1, a gas sensor 100 has a closed-end cylindrical gas sensing element (an oxygen detection element) 200 having a leading end portion 3. A rod-shaped heater 101 is inserted into a cylindrical portion of the gas sensing element 200.

The gas sensing element 200 includes a solid electrolytic element 1 that contains zirconia as a main component and that exhibits oxygen ion conductivity. The leading end portion 3 side of the solid electrolytic element 1 is provided with a detecting area where an outer electrode 5 described below is formed (a lower side in the drawing). The detecting area is provided with a flange 2 projecting outside in a radial direction at a rear end side thereof (an upper side in the drawing).

The outer electrode 5 is made of, for instance Pt or a Pt alloy, by means of plating, such as electroless plating. The outer electrode 5 is formed on an exterior side of the solid electrolytic element 1. The outer electrode 5 is electrically connected to a lead electrode (not shown) which is a path for a signal from the outer electrode 5 to the rear end side of the gas sensing element 200. On an exterior side of the outer electrode 5, a protective layer (not shown) formed from a ceramic sprayed layer, such as a spinel, is provided. On an inner side of the solid electrolytic element 1 is also provided with an inner electrode (not shown) made of, for instance, Pt or a Pt alloy, similar to the outer electrode 5.

The gas sensing element 200 is hermetically held in a through hole 171 of a cylindrical metal shell 105. The flange 2 of the gas sensing element engages a holder 102 made of insulating ceramic. For hermetic sealing, ceramic powder 104 formed from talc is provided at the rear end of the flange 2 and a sleeve 103 is placed at the rear end side of the ceramic powder 104.

As shown in FIG. 1 the gas sensing element 200 extends along an axis (the vertical direction in FIG. 1). In the present specification, a portion of the gas sensing element 200 at the side of the leading end portion 3 (a downward direction in FIG. 1) along the axis of the gas sensing element 200 is called a leading end side, and a portion of the same at the opposite side along the axis (in an upward direction in FIG. 1) is called a rear end side.

The metal shell 105 has a screw portion 106 and a tool engagement portion 107 for attaching the gas sensor 100 to a mounting portion of an exhaust pipe, or the like. The metal shell 105 also has a protector connecter portion 109 connected to a protector 108 by means of laser welding. The protector 108 is attached so as to cover a portion of the leading end portion 3 of the gas sensing element 200. When in use, a leading end side of the gas sensor 100 relative to the screw portion 106 is positioned in the engine, such as an exhaust pipe, and a rear end side of the gas sensor 100 relative to the screw portion 106 is positioned in the external atmosphere.

Meanwhile, a rear end portion 110 of the metal shell 105 is crimped and hermetically held with a ring packing 111 sandwiched between the rear end portion 110 and the sleeve 103. A leading end portion 114 of a cylindrical metal tube 113 is fixed to a connecter portion 112 on a rear end side of the tool engagement portion 107 by means of laser welding. A main part of the housing in the gas sensor 100 is built from the metal shell 105 and the metal tube 113.

A sealing member; for instance, a grommet 120 made from rubber, or the like, is fitted and crimped (by metal tube 113) into an opening on the rear end side of the metal tube 113; namely, a substantial rear end side opening of the housing, to thereby seal the opening.

A filter 210 is provided in the center of the grommet 120. The filter 210 introduces the atmosphere into the metal tube 113 and prevents droplet from intruding into the metal tube 113. A separator 122 formed from insulating alumina ceramic is provided at a leading end side of the grommet 120. Sensor output lead wires 130 and 131 and heater lead wires 132 and 133 are provided so as to penetrate through the separator 122 and the grommet 120.

The separator 122 has a substantially cylindrical shape. The separator 122 includes a rear end portion 123, the leading end portion 124, and a flange portion 125 interposed between the rear end portion 123 and the front end portion 124. The diameter of the flange portion 125 is larger than that of the rear end portion 123 and the front end portion 124. The flange portion 125 has a tapered surface 126 and a step surface 127. The tapered surface 126 is provided at an area of the separator 122 between the flange portion 125 and the rear end portion 123. The tapered surface 126 is widely tapered toward the leading end side (the downward direction in the drawing). Meanwhile, the step surface 127 is a stair-like step provided at an area between the flange portion 125 and the leading end portion 124.

The separator 122 supports a connector 141 of a first sensor terminal 140, a connector 241 of a second sensor terminal 240, and heater terminals 340, 341 therein. In this configuration, the connectors 141, 241 and the heater terminals 340, 341 are insulated from each other by the separator 122.

The first sensor terminal 140 has the connector 141, a branch 142, and an insert terminal 143 that are integrally formed. The connector 141 electrically connects the first sensor terminal 140 with the sensor output lead wire 130 by gripping a core wire of the sensor output lead wire 130.

The branch 142 holds the first sensor terminal 140 in the separator 122 by elastically contacting a holding hole of the separator 122. The insert terminal 143 is inserted into a cylindrical portion of the gas sensing element 200, to thereby establish electrical conduction with the inner electrode (a reference electrode). Also, the insert terminal 143 encloses the heater 101.

When inserted into the cylindrical portion of the gas sensing element 200, since the insert terminal 143 presses the heater 101, an axis line of the heater 101 is decentered with respect to the axis along which the gas sensing element 200 extends. The heater 101 thereby contacts a cylindrical interior wall (the inner electrode) of the gas sensing element 200, to make a heater contact portion 201. A heating portion 161 is provided which corresponds to the heater contact portion 201.

Since the heater contact portion 201 contacts a cylindrical interior wall of the gas sensing element 200, heat energy is concentrated in a smaller volume. This heat energy concentration is effective for shortening an activation time of the gas sensor 100. In the gas sensing element 200, an area of the gas sensing element 200 at the heater contact portion 201 serves as a detecting section 202.

Meanwhile, the second sensor terminal 240 has the connector 241, a branch 42, and a grip 243 that are integrally formed. The connector 241 electrically connects the second sensor terminal 240 with the sensor output lead wire 131 by gripping a core wire of the sensor output lead wire 131. The branch 242 holds the second sensor terminal hardware 240 in the separator 122 by elastically contacting a holding hole of the separator 122. Further, the grip 243 elastically grips in the vicinity of an outer periphery of the rear end side of the gas sensing element 200.

The heater 101 is a rod-shaped ceramic heater and includes a heating portion 161. The heating portion 161 includes a core mainly made from alumina and a resistance heating element formed around the core. When the heater 101 is energized through heater terminals 340 and 341 and the heater lead wires 132, 133 brazed to electrodes pads 163, 164, the leading end portion of the gas sensor element 200 is heated.

The metal tube 113 is made of metal and has a substantially cylindrical shape. As described above, the metal tube 113 has a first tube portion 115 and a second tube portion 116. The first tube portion 115 has the leading end portion 114 joined to the metal shell 105. The second tube portion 116 is located at a rear end side than is the first tube portion 115 and smaller in diameter than the first tube portion 115. Inner projections 117 are formed at a radial middle portion of the second tube portion 116. The inner projections 117 are equivalently formed in a peripheral direction of the second tube portion 116. Each inner projection 117 protrudes in an inner radial direction while the apex of the inner projection 117 forms a rectangular surface. Each inner projection 117 has an inclined surface 118 at the leading end side of the apex. The inclined surface 118 of individual ones of the inner projections 117 contacts the tapered surface 126 of the separator 122.

A spring clasp 150 is attached around the leading end portion 124 of the separator 122. In addition to having a cylindrical metal sleeve 151, spring clasp 150 has four elastic holders 152 that are formed integrally with the metal sleeve 151 at a rear end of the metal sleeve 151.

The elastic holders 152 are respectively placed at four points with equal intervals therebetween along a peripheral direction of the metal sleeve 151. Each of the elastic holders 152 extends in a radial inner direction from the rear end of the metal sleeve 151 and gradually bends toward the leading end side so as to extend along the axial direction. When the spring clasp 150 is attached to the leading end portion 124 of the separator 122, the elastic holder 152 becomes elastically deformed and pushes the leading end portion 124 of the separator 122 in the radial inner direction so as to hold the spring clasp 150 at the leading end portion 124.

The gas sensor 100 of above described configuration is used while a leading end side (a lower side in FIG. 1) of the gas sensor ahead of the screw (threaded) portion 106 is positioned in the exhaust pipe while a rear end side (an upper side in FIG. 1) of the gas sensor behind the screw portion 106 remains in the external atmosphere. The gas sensing element 200 is heated and activated by the heater 101 disposed in the cylindrical portion of the gas sensing element 200. The atmosphere as a reference gas is introduced into the metal tube 113 through the filter 210 and guided into the gas sensing element 200. Meanwhile, an exhaust gas is guided to the outside of the gas sensing element 200 through air holes 187 of the protector 108.

Accordingly, electromotive force is generated between the outer electrode and the inner electrode when there is a difference between oxygen concentration at the exterior surface of the gas sensing element 200 and the interior surface of the gas sensing element 200. This electromotive force due to a difference in oxygen concentration is extracted as a detection signal relating to the concentration of oxygen in the exhaust gas. Thus, the concentration of oxygen in the exhaust gas is detected.

FIG. 2 shows an enlarged view of the leading end portion of the gas sensor 100 having the above described configuration. As illustrated, in the first embodiment, the leading end of the gas sensing element 200 protrudes from the leading end of the metal shell 105 by 4 mm. Therefore, the entirety of each of the heater contact portion 201 and the detecting portion 202 remains within the metal shell 105 and does not protrude out of the leading end of the metal shell 105.

The protector 108 comprises an outer protector 186 fitted around the leading end side of the metal shell 105 radially and from outside the leading end side, and an inner protector 181 disposed inside the outer protector 186 and spaced apart from the outer protector 186 in the radial direction.

The outer protector 186 has an outer tubular portion 188, an outer tapered portion 189, and an outer bottom portion 190. The outer tubular portion 188 extends toward the leading end side and has a rear end that is fitted around the leading end side of the metal shell 105 radially and from outside the leading end side. The outer tapered portion 189 is jointed to a leading end of the outer tubular portion 188. The outer bottom portion 190 is jointed to the outer tapered portion 189. A plurality of air holes 187 are formed in the outer tubular portion 188 along a circumferential direction.

The inner protector 181 has an inner tubular portion 184 disposed opposite the outer tubular portion 188, an inner tapered portion 185 jointed to the leading end of the inner tubular portion 184, and an inner bottom portion 192 joined to the inner tapered portion 185.

A plurality of entrance air vents 182 are formed along the circumferential direction of the inner tubular portion 184. The entrance air vents 182 are provided between a base end side (a rear end side) of the inner tubular portion 184 and the metal shell 105. Specifically, as shown in FIG. 3, the inner protector 181 has intermittent flanges 193 that radially project outside from a rear end of the inner tubular portion 184 and that are intermittently arranged in the circumferential direction. The spaces between the intermittent flanges 193 serve as the entrance air vents 182. An exit air hole 183 is formed in the inner bottom portion 192. The inner bottom portion 192 and the outer bottom portion 190 are in contact with each other, and the exit air hole 183 extends to the outer bottom portion 190.

As shown in FIG. 2, the leading end portion of the metal shell (housing) 105 has an expanding portion 501 having the through hole 171 whose radial diameter increases toward the leading end. In the first embodiment, the tapered portion 501 is made in a tapered fashion.

The gas sensor 100 is fixedly inserted into an exhaust pipe or the like while the rear end of the gas sensor 100 assumes an upper position. In the exhaust pipe, there is horizontal circulation of exhaust gas as illustrated by the horizontal arrow in FIG. 2. In this case, the exhaust gas flows from the air holes 187 of the outer protector 186 into space between the outer protector 186 and the inner protector 181 (the space between the outer tubular portion 188 and the inner tubular portion 184). In addition to containing an exhaust gas component, the exhaust gas contains moisture, or the like. The moisture also flows into the space between the outer tubular portion 188 and the inner tubular portion 184 through one of the air holes 187. Since the moisture is heavier than the exhaust gas component, the moisture exits outside of the space between the outer protector 186 and the inner protector 181 while being guided by another of the air holes 187 opposite to the one of the air holes 187.

Meanwhile, since the exhaust gas component is lighter than the moisture, the exhaust gas component goes up in the space between the outer protector 186 and the inner protector 181 and flows into the inner protector 181 through the entrance air vents 182. As indicated by an arrow in the drawing, since there is a flow of the exhaust gas running along the outer tapered portion 189 and passing outside the exit air hole 183, a negative pressure develops in the vicinity of the exit air hole 183. Because of the negative pressure, the exhaust gas flowing into the inner protector 181 is discharged to the outside from the exit air hole 183.

By virtue of such a flow of the exhaust gas, the exhaust gas is supplied to the detecting portion 202 of the gas sensing element 200, where the concentration of oxygen in the exhaust gas is detected. At this time, in the first embodiment, the entire detecting portion 202 of the gas sensing element 200 remains within the metal shell 105 and does not project from the leading end of the metal shell 105. Since the exhaust gas is introduced into the metal shell 105 along the expanding portion 501 of the leading end portion of the metal shell (housing) 105, this configuration can prevent deterioration of responsiveness.

As shown in FIG. 2, a preferred relationship between the shape of the expanding portion 501 and the position of the detecting portion 202 of the gas sensing element 200 is that at least a portion of the heater contact portion 201 corresponding to the detecting portion 202 is positioned on the leading end side of an imaginary line connecting the rear end and front end of the expanding portion 501 of the through hole 171 (indicated by an alternate long and short dash line in the drawing). In FIG. 2, since the expanding portion 501 is formed so as to have a tapered profile, the imaginary line runs along the tapered surface in this exemplary embodiment. The gas can quickly arrive at the vicinity of the detecting portion 202 of the gas sensing element 200 (the heater contact portion 201) by means of the flow of gas running along an inner peripheral surface of the expanding portion 501. Thus, deterioration of responsiveness can be prevented.

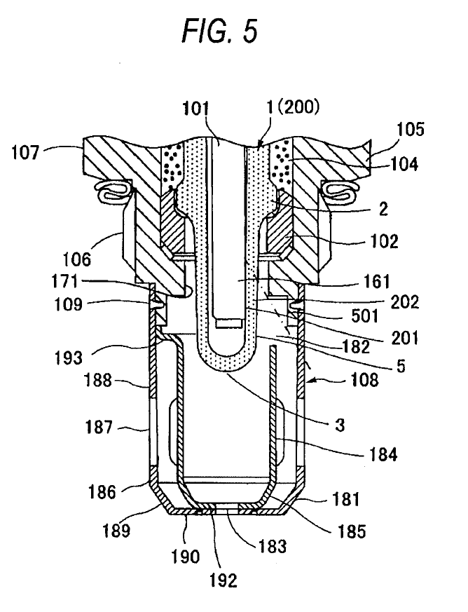

As shown in FIG. 5, even in a case where the expanding portion 501 does not have a tapered profile and is formed instead in a step shape, the essential requirement is that at least a portion of the heater contact portion 201 is positioned on the leading end side of the imaginary line (indicated by the alternate long and short dash line in the drawing) connecting the rear end and the front end of the expanding portion 501 of the through hole 171. As shown in FIG. 2, the through hole 171 of the expanding portion 501 is more preferably formed so as to have a tapered profile that gradually expands in diameter toward the leading end. The reason is that the taper profile keeps the amount of the gas flowing into the expanding portion 501 and maintains the speed of the gas flow before the gas arrives at the detecting portion 202 of the gas sensing element 200. Thus, deterioration of responsiveness can be prevented.

At least a portion of the heater contact portion 201 (the entirety of the same in the first embodiment) is positioned so as to be closer to the leading end side relative to the rear end of the expanding portion 501 of the through hole 171. Hence, a large amount of gas can quickly arrive at the detecting portion 202 of the gas sensing element 200 by means of the flow of gas running along the expanding portion 501 of the through hole 171. Thus, deterioration of responsiveness can be further prevented.

At least some of the entrance air vents 182 are provided at a radially inner side of the imaginary line connecting the rear end and the leading end of the expanding portion 501 of the through hole 171. A large amount of the gas that passes through the space between the outer tubular portion 188 and the inner tubular portion 184 quickly flows toward the expanding portion 501 without substantial influence of the leading end of the metal shell 105. Consequently, a large amount of gas can quickly arrive at the detecting portion 202 of the gas sensing element 200. Thus, deterioration of responsiveness can be prevented.

### EXAMPLES

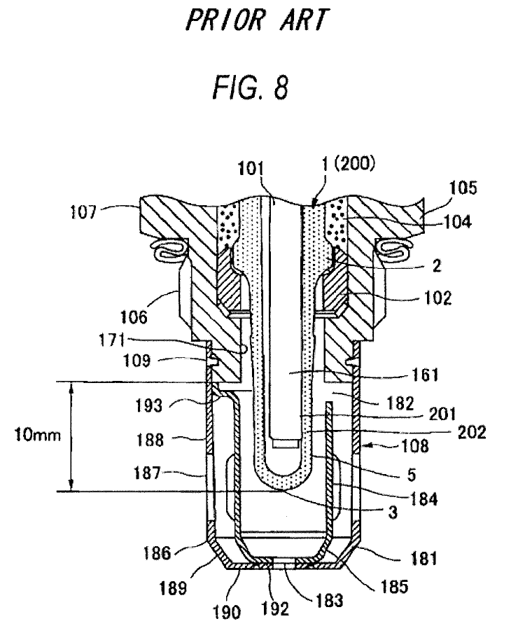

FIG. 8 shows an enlarged view of the configuration of the leading end of a related-art gas sensor. As illustrated, in the related-art gas sensor, the leading end portion of the gas sensing element 200 is long, and the leading end of the gas sensing element 200 projects from the leading end of the metal shell 105 by about 10 mm. The heater contact portion 201 projects from the leading end of the metal shell 105. The related-art gas sensor is not equipped with the expanding portion 501.

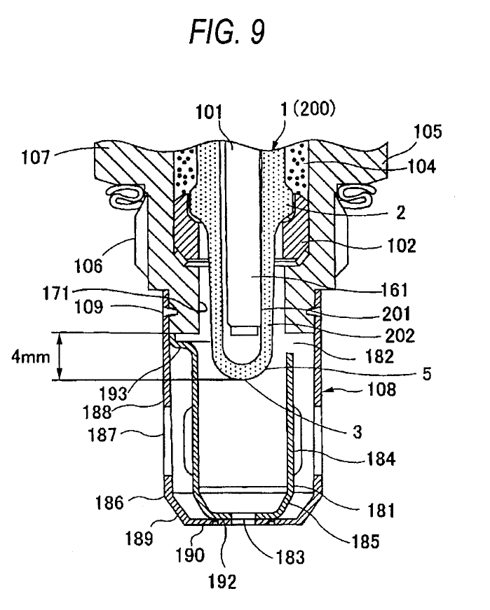

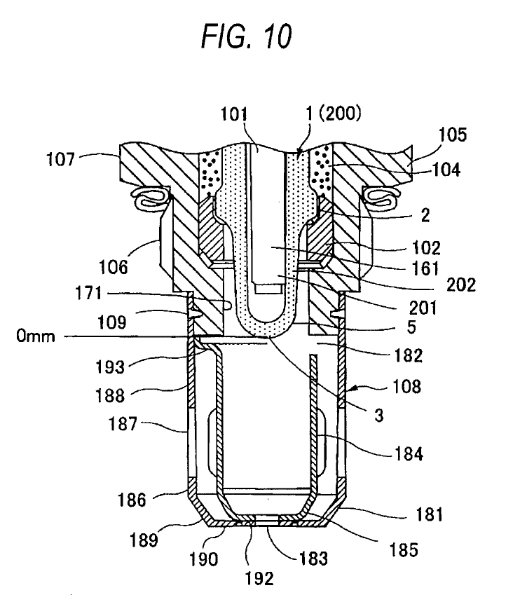

In such a configuration, responsiveness was measured for three cases. In a first case, the length of the leading end portion of the gas sensing element 200 was reduced such that the leading end of the gas sensing element 200 was arranged so as to project from the leading end of the metal shell 105 by 4 mm as shown in FIG. 2 (a first comparative example). The first case is illustrated in FIG. 9. In the second case, the leading end of the gas sensing element 200 was arranged so as to project from the leading end of the metal shell 105 by 2 mm (a second comparative example). In the third case, as shown in FIG. 10, the leading end of the gas sensing element 200 was arranged so as to project from the leading end of the metal shell 105 by 0 mm (i.e., where the leading end of the gas sensing element was arranged so as not to project from the leading end of the metal shell) (a third comparative example).

Responsiveness was measured by attaching the gas sensor to an exhaust pipe of a 4-cycle engine having a piston displacement of 2000 cc and measuring a value output from the gas sensor when the engine was driven at 2000 rpm. In each comparative example, the gas sensor was attached to the exhaust pipe at a location where an exhaust gas temperature came to about 450° C. During measurement, the air fuel ratio λ was controlled so as to switch the condition at two seconds between a rich mixture (λ=0.97) and a lean mixture (λ=1.03) where λ=1 is the condition where a theoretical air fuel ratio is 14.7. TRS is defined as a period during which the output of the oxygen sensor of the comparative examples changes to a value corresponding to λ=1 after the condition is switched from a rich mixture to a lean mixture. TLS is defined as a period during which the output of the oxygen sensor of the comparative examples changes to a value corresponding to λ=1 after the condition is switched from the lean mixture to the rich mixture.

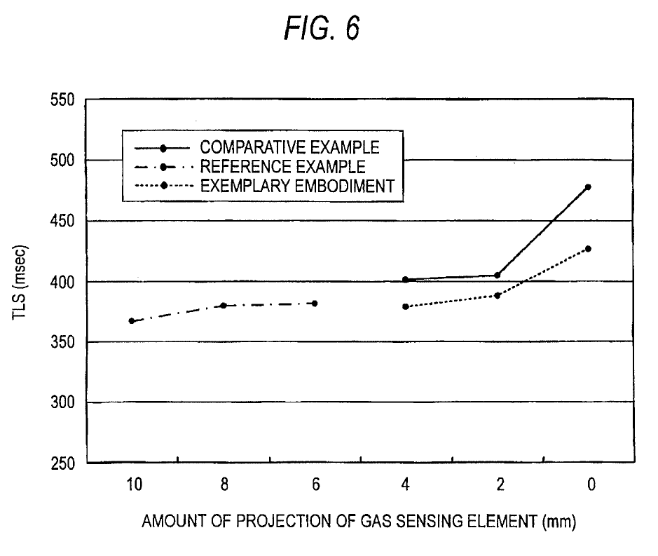

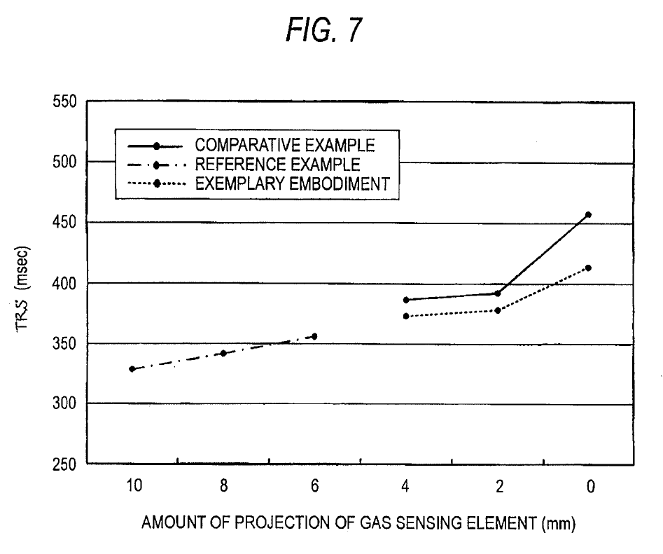

As seen from the graph shown in FIG. 6 in which the vertical axis represents TLS (msec.) and the horizontal axis represents the projection amount of the gas sensing element (the length of a projection) (mm) and the graph shown in FIG. 7 in which the vertical axis represents TRS (msec.) and the horizontal axis represents the amount of projection of the gas sensing element (the length of a projection) (mm), when the projection amount (the length of a projection) of the related-art gas sensor was reduced to 4 mm or less, the response time became longer, and deterioration of responsiveness noticeably appeared, as shown in FIGS. 9 and 10. FIGS. 6 and 7 also show data pertaining to reference examples achieved when the projection amount (the length of the projection) of the related-art gas sensor was set to 10 mm, 8 mm, and 6 mm.

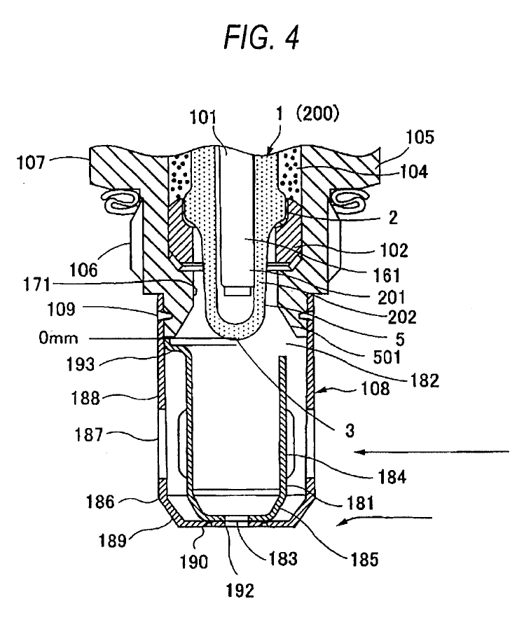

In the exemplary embodiment shown in FIG. 2, responsiveness was measured for three cases. In the first case, the projection amount (the length of the projection) was set to 4 mm (a first example). In the second case, the projection amount (the length of the projection) was set to 2 mm (a second example). In the third case, the projection amount (the length of the projection) was set to 0 mm as shown in FIG. 4 (i.e., a case where the gas sensing element was arranged so as not to project from the leading end of the metal shell) (a third example). As shown in FIGS. 6 and 7, when the measurement results are compared with those of to the three comparative examples, deterioration of responsiveness can clearly be prevented.

As shown in FIG. 2, when the projection amount (the length of the projection) is set to 4 mm or less, the location of the heater contact portion (detecting portion) 201 where the heater 101 contacts the gas sensing element 200 is on the rear end side (inside) with reference to the leading end of the metal shell 105. Therefore, in the foregoing three comparative examples, substantial deterioration of responsiveness is apparent.

In the first exemplary embodiment, the outer electrode 5 is an exemplary embodiment of detection electrode; the air hole 187 is an exemplary embodiment of the outer air hole; the entrance air vent 182 is an exemplary embodiment of the inner air vent.

As described above, the gas sensor 100 of the first embodiment makes it possible to shorten the gas sensing element 200 while preventing deterioration of responsiveness and reducing manufacturing cost.

### Second Exemplary Embodiment

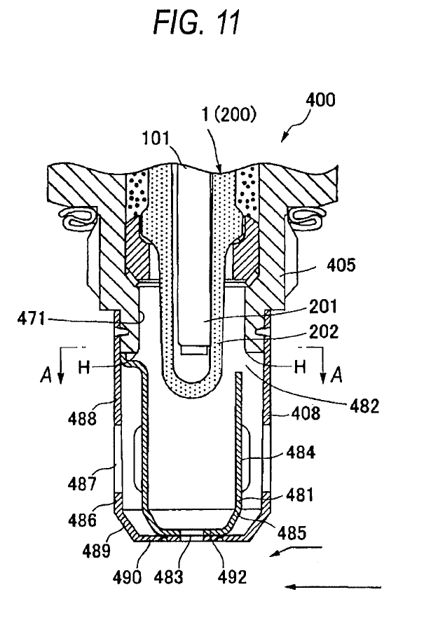

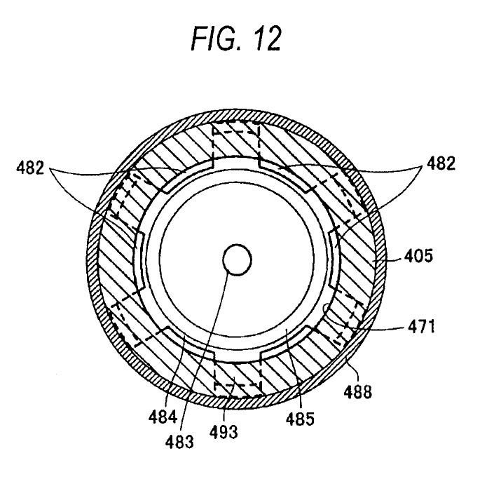

A gas sensor 400 of a second embodiment of the present invention is now described. FIG. 11 is a longitudinal cross sectional view showing, in an enlarging manner, the configuration of a principal section of the gas sensor 400, and FIG. 12 is a lateral cross-sectional view taken along line A-A shown in FIG. 11. The gas sensing element 200 and the heater 101 are omitted from FIG. 12. Although the gas sensor 400 of the second embodiment differs from the gas sensor 100 of the first embodiment in terms of the structure of a metal shell 405 and the structure of a protector 408, the same elements as those employed in the gas sensor 100 are used for the other elements of the gas sensor 400. Therefore, the elements common between the gas sensors 100 and 400 are assigned the same reference numerals in the drawing, and their explanations are omitted or simplified.

In the second embodiment, the leading end of the gas sensing element 200 projects from the leading end of the metal shell 405 by 4 mm. Therefore, the entirety of the heater contact portion 201 and the entirety of the detecting portion 202 do not project from the leading end of the metal shell 405 and remain accommodated in the metal shell 405.

The protector 408 has an outer projector 486 fitted around a leading end side of the metal shell 405 and an inner protector 481 provided in the outer protector 486 while being spaced apart from the outer protector 486.

The outer protector 486 has an outer tubular portion 488, an outer tapered portion 489, and an outer bottom portion 490. The outer tubular portion 488 extends toward the front end side and its rear end is fitted around the leading end side of the metal shell 405. The outer tapered portion 489 joins a leading end of the outer tubular portion 488. The outer bottom portion 490 joins the outer tapered portion 489. A plurality of air holes 487 are formed in the outer tubular portion 488 along a circumferential direction.

The inner protector 481 has an inner tubular portion 484 disposed opposite the outer tubular portion 488, an inner tapered portion 485 jointed a leading end of the inner tubular portion 484, and an inner bottom portion 492 joined to the inner tapered portion 485.

A plurality of entrance air vents 482 are formed, along the circumferential direction, in the inner protector 481 between a base end side (a rear end side) of the inner tubular portion 484 and the metal shell 405. Specifically, the inner protector 481 has the same configuration as that shown in FIG. 3, and has intermittent flange portions 493 (see FIG. 12). The intermittent flange portions 493 radially project outside from a rear end of the inner tubular portion 484 and are intermittently arranged in the circumferential direction. Space between the intermittent flange portions 493 serves as the entrance air vents 482. An exit air hole 483 is formed in an inner bottom portion 492. The inner bottom portion 492 and the outer bottom portion 490 remain in contact with each other, and the exit air hole 483 extends to the outer bottom portion 490.

As shown in FIG. 11, in the second embodiment, the entrance air vent 482 is positioned inside in the radial direction with reference to an intersection point H of a through hole 471 of the metal shell 405 and a leading end (a leading end face) of the metal shell 405. As shown in FIG. 12, the entrance air vents 482 are thereby exposed radially inside with reference to the through hole 471 of the metal shell 405. When a chamfer is provided so as to link the through hole 471 of the metal shell 405 to the leading end face of the metal shell 405, the intersection point H denotes a point of intersection of the leading end face of the metal shell 405 and the through hole 471 (including the chamfer).

The gas sensor 400 is fixedly inserted into an exhaust pipe, or the like, which effects horizontal circulation of exhaust gas in such manner that a rear end of the gas sensor 400 assumes an upper position, as indicated by the illustrated arrow. In this case, the exhaust gas flows from the air holes 487 of the outer protector 486 into the space between the outer protector 486 and the inner protector 481 (the space between the outer tubular portion 488 and the inner tubular portion 484). In addition to containing an exhaust gas component, the exhaust gas contains moisture, or the like. Since moisture, or the like, is heavier than the exhaust gas component, the moisture exits outside of the space between the outer protection tube 486 and the inner protection tube 481, while being guided by way of the air holes 487 provided on an opposite side of the outer protection tube 486.

Meanwhile, the exhaust gas component is lighter than the moisture and hence goes up between the outer protector 486 and the inner protector 481, thereby flowing into the inner protector 481 from the entrance air vents 482. As indicated by an arrow in the drawing, a flow of exhaust gas running along the outer tapered portion 489 forms outside the exit air hole 483. A negative pressure develops in the vicinity of the exit air hole portion 483 so that exhaust gas flowing into the inner protector 481 is discharged to the outside from the exit air hole 483.

By virtue of such a flow of the exhaust gas, the exhaust gas is supplied to the detecting portion 202 of the gas sensing element 200, where the concentration of oxygen in the exhaust gas is detected. At this time, in the second embodiment, the entire detecting portion 202 of the gas sensing element 200 does not project from the leading end of the metal shell 405 and remains accommodated in the metal shell 405. However, so long as the gas sensing element is configured in such a way that at least some of the entrance air vents 482 are provided radially inside with reference to the intersection point H of the through hole 471 and the leading end of the metal shell 405, the large amount of gas passing through the space between the outer tubular portion 488 and the inner tubular portion 484 quickly flows into the through hole 471 of the metal shell 405 without being hindered by the metal shell 405. Therefore, the flow of the gas easily arrives at the vicinity of the detecting portion 202 of the gas sensing element 200 (the heater contact portion 201) inserted into the metal shell 405, so that deterioration of responsiveness can be prevented.

While the present invention has been shown and described with reference to certain exemplary embodiments thereof, it will be understood by those skilled in the art that various changes in form and detail may be made therein without departing from the spirit and scope of the invention as defined by the appended claims.

For instance, in the first and second embodiments, the air holes 187 and 487 are provided in the respective outer tubular portions 188 and 488. However, the air holes are not limited to the outer tubular portions but may also be provided in the respective outer tapered portions 189 and 489. In the first and second embodiments, the heater 101 is positioned off center with reference to the center axis line of the gas sensing element 200, whereby the heater 101 is in contact with the gas sensing element 200. However, the heater 101 and the gas sensing element 200 are not limited to such a configuration. For instance, a leading end edge of the heater 101 may also be in contact with an inner peripheral surface of the leading end portion 3 of the gas sensing element 200 while the center axis line of the sensing element 200 and the center axis line of the heater 101 may be held in alignment with each other. Although the entirety of the heater contact portion 201 (the detecting portion 202) is accommodated in the metal shell 105 and the meal shell 405 in the first and second embodiments, a portion of the heater contact portion 201 (the detecting portion 202) may also project from the leading end of each of the metal shells 105 and 405.

This application is based on Japanese Patent Application Nos. 2009-100049 filed Apr. 16, 2009 and JP 2009-124190 filed May 22, 2009, the disclosures of which are incorporated herein by reference in their entirety.

What is claimed is:

1. A gas sensor extending along an axial direction from a rear end side to a leading end side thereof, the gas sensor comprising:
a cylindrical housing having a through hole, the through hole including a radially expanding portion at a leading end thereof;
a cylindrical gas sensing element inserted into the housing, having a closed leading end, and provided with a sensor electrode on an outer surface of the leading end side of the gas sensing element;
a bar shaped heater inserted into the gas sensor, and including a contact portion where the heater contacts an inner surface opposite the outer surface of the leading end side of the gas sensing element, the contact portion and the leading end of the housing positioned in this order with respect to the axial direction from the rear end side to the leading end side;
a protector fixed on the leading end side of the housing and having a cylindrical outer protector including an outer air hole and a cylindrical inner protector being positioned within the outer protector and spaced apart from the outer protector in the radial direction; and
an inner air vent provided between the inner protector and the leading end of the housing, the inner air vent guiding gas entering a space between the outer and inner protectors into the through hole and into the inner protector.

2. The gas sensor according to claim 1, wherein the gas sensing element includes a solid electrolyte and the sensor electrode is provided on an outer surface of the leading end side of the solid electrolyte.

3. The gas sensor according to claim 1, wherein the contact portion of the heater contacts the inner surface opposite the outer surface of the leading end side of the gas sensing element through an intermediate member.

4. The gas sensor according to claim 1, wherein at least a portion of the inner air vent is positioned at a radially inner side of an imaginary line defined between a leading end of the expanding portion and a rear end of the expanding portion when the gas sensor is viewed in a cross section parallel to the axial direction.

5. The gas sensor according to claim 1, wherein at least a portion of the contact portion is positioned at a leading end side of an imaginary line defined between a leading end of the expanding portion and a rear end of the expanding portion when the gas sensor is viewed in a cross section parallel to the axial direction.

6. The gas sensor according to claim 1, wherein the rear end of the expanding portion and at least a portion of the contact portion are positioned in this order with respect to the axial direction from the rear end side to the leading end side when the gas sensor is viewed in a cross section parallel to the axial direction.

7. The gas sensor according to claim 1, wherein the through hole within the expanding portion gradually expands in the radial direction along the axial direction from the rear end side to the leading end side.

8. A gas sensor extending along an axial direction from a rear end side to a leading end side, the gas sensor comprising:
a cylindrical housing having a through hole;
a cylindrical gas sensor inserted into the housing, having a closed leading end, and provided with a sensor electrode on an outer surface of the leading end side of the gas sensing element;
a bar shaped heater inserted into the gas sensor, and including a contact portion where the heater contacts an inner surface opposite the outer surface of the leading end side of the gas sensing element, the contact portion and the leading end of the housing positioned in this order with respect to the axial direction from the rear end side to the leading end side;
a protector fixed on the leading end side of the housing and having a cylindrical outer protector including an outer air hole and a cylindrical inner protector being positioned within the outer protector and spaced apart from the outer protector in the radial direction; and
an inner air vent provided between the inner protector and the leading end of the housing, the inner air vent guiding gas entering a space between the outer and inner protectors into the through hole and into the inner protector,
wherein at least a portion of the inner air vent is provided at a radially inner side of a cross point between the through hole and the leading end of the housing when the gas sensor is viewed in a cross section parallel to the axial direction.

9. The gas sensor according to claim 8, wherein the gas sensing element includes a solid electrolyte and the sensor electrode is provided on an outer surface of the leading end side of the solid electrolyte.

10. The gas sensor according to claim 8, wherein the contact portion of the heater contacts the inner surface opposite the outer surface of the leading end side of the gas sensing element through an intermediate member.

11. The gas sensor according to claim 1, comprising:
a gas flow hole provided at a leading end side of the inner protector.

12. The gas sensor according to claim 1, comprising:
the outer air hole provided at a leading end side of the leading end of the gas sensing element.

13. The gas sensor according to claim 8, comprising:
a gas flow hole provided at a leading end side of the inner protector.

14. The gas sensor according to claim 8, comprising:
the outer air hole provided at a leading end side of the leading end of the gas sensing element.
